(19) United States
(12) Patent Application Publication
(10) Pub. No.: US 2026/0102535 A1
(43) Pub. Date: Apr. 16, 2026

(54) ABSORBENT ARTICLES INCLUDING HIPE FOAM IMPARTED WITH COLOR, AND METHOD OF MANUFACTURE

(71) Applicant: THE PROCTER & GAMBLE COMPANY, Cincinnati, OH (US)

(72) Inventors: Digvijay RAWAT, Cincinnati, OH (US); Maxwell Joseph WINGERT, Liberty Township, OH (US); Nicholas David VETTER, Cleves, OH (US)

(21) Appl. No.: 19/356,429

(22) Filed: Oct. 13, 2025

Related U.S. Application Data
(60) Provisional application No. 63/707,774, filed on Oct. 16, 2024.

(51) Int. Cl. A61L 15/24; A61F 13/472; A61F 13/53; A61F 13/531; A61F 13/84; C08J 9/00

(52) U.S. Cl. CPC A61L 15/24; A61F 13/472; A61F 13/531; A61F 13/8405; C08J 9/0004; A61F 2013/530817; A61F 2013/8497

(57) ABSTRACT

A method for manufacturing an open cell HIPE foam structure imparted with color is disclosed. The method may include steps of: suspending dry pigment particles in a suspending medium to create a first suspension; providing an oil phase component blend including at least one monomer; providing an aqueous phase component solution; providing an initiator solution; blending the first suspension with the oil phase component blend to create a second suspension; blending the second suspension with the aqueous phase component solution and the initiator solution in water:oil ratio of at least 2.85:1 by volume, to create a water-in-oil HIPE having a continuous phase comprising the second suspension; and polymerizing monomer components of the oil phase component blend. The associated pigment-in-monomer second suspension, resulting open cell HIPE foam imparted with color. Absorbent articles made therefrom are also disclosed.

### CROSS REFERENCE TO RELATED APPLICATIONS

This application claims the benefit, under 35 U.S.C. § 119(e), to U.S. Provisional Application No. 63/707,774 filed Oct. 16, 2024, the entire disclosure of which is fully incorporated by reference herein.

### FIELD

The present disclosure is generally directed to high internal phase emulsion foams, and more particularly, to high internal phase emulsion foams imparted with color and methods of manufacture of absorbent articles manufactured therefrom.

### BACKGROUND

Currently, certain types of feminine hygiene pads (also called “sanitary napkins”) available in the market have an absorbent component formed of open-celled foam. The absorbent foam in these pads can be manufactured via polymerization of one or more monomers constituting the oil phase of a high internal phase, water-in-oil emulsion.

A high internal phase emulsion (“HIPE”) for purposes herein is a two-phase water-in-oil emulsion in which the water-to-oil ratio is greater than 2.85:1, i.e., greater than 74 percent dispersed aqueous phase and less than 26 percent continuous monomer/oil phase (by volume). Due to this relatively high water-to-oil ratio, upon emulsification sufficient to result in a foam of the type contemplated herein (i.e., average cell size 1 to 300 micrometers), the dispersed aqueous phase will have the form of droplets forced into polyhedral forms as a result of close crowding, separated by thin film walls formed of the continuous monomer/oil phase. Upon subsequent processing, the monomer(s) of the continuous oil phase is/are polymerized, resulting in solid, interconnected, fine “struts” in regions where groups of three or more droplets are adjacent, that create the structure of the foam. Directly between pairs of adjacent droplets and between or circumscribed by the struts, the process causes the thin film walls to open, thus creating open cells and resulting in a structure of interconnected struts that defines an open-celled and liquid permeable foam.

The continuous (oil) phase of the HIPE includes the monomer(s) to be polymerized, and an emulsifier to help stabilize the HIPE prior to curing. The oil phase may also include one or more photoinitiators, in the event it is desired to effect polymerization via application of light energy to the HIPE. The monomer component(s) may be included in an amount of from about 80% to about 99% by weight of the oil phase. The emulsifier component, which is soluble in the oil phase and suitable for forming a stable water-in-oil emulsion, may be included in the oil phase in an amount of from about 1% to about 20% by weight of the oil phase.

The foam resulting from the process typically has a substantially white color under ordinary midday clear daylight conditions (illuminance 10,000 lux and higher), resulting in part from the relative colorlessness of the polymerized material, and in part from the cellular structure in which incident light is reflected or refracted from or through the structure in many different directions.

It has been learned that absorbent articles such as (but not necessarily limited to) disposable diapers, disposable absorbent pants, and feminine hygiene pads may be better received and/or appreciated by consumers when they include elements of color in their appearance. In some cases this greater appreciation may simply be a matter of esthetics. In some cases this greater appreciation may be a result of consumer impressions that components that have particular coloration are more effective, e.g., are more absorbent, or include particularly effective/absorbent materials. In some examples coloration may be imparted to absorbent components to signal particular absorbency or efficacy of particular components, materials and/or particular locations within the pad.

However, it is believed that, to date, persons working within the art have not appreciated that color imparted to a HIPE foam can be beneficial from a consumer appreciation perspective, and/or, alternatively, that a HIPE foam can be successfully imparted with coloration.

Formation of a HIPE of sufficient stability, and maintenance of the emulsion without undesirable and/or unacceptable level of coalescence of suspended aqueous phase droplets through the time that the monomer/oil phase is subjected to a polymerization process, is typically sensitive with respect to chemistry and process. Introduction of typical color-imparting materials such as dyes and pigments to the component blend can introduce undesired disruptions in the interfaces between dispersed and continuous phases, or undesired chemical reactions, thereby inhibiting or altering emulsion formation or resulting in an emulsion of insufficient stability to remain intact through the polymerization process. Further, any foam produced with intended, imparted coloration may not be sufficiently colorfast, such that the foam will bleed dissolved coloring materials upon contact with aqueous fluid, or shed particulate coloring materials. These events will generally have undesirable consequences such as staining of a wearers' skin and/or or wearer/consumer perceptions of poor quality.

Thus, there is a need for an absorbent article such as (but not limited to) a disposable diaper, disposable absorbent pant or feminine hygiene pad, with a foam component or a portion thereof imparted with coloration. There is a further need for such a foam component or a portion thereof to be satisfactorily colorfast.

### SUMMARY

To solve the problems advanced above, the present disclosure provides an open-cell foam comprising a structure of interconnected struts formed of polymeric material and defining open cells, resulting from polymerization of a continuous phase of a high internal phase water-in-oil emulsion, the struts having pigment particles at least partially encapsulated or embedded within the polymeric material thereof.

Also described herein is a method for manufacturing an open cell foam structure comprising a polymerized continuous phase of a high internal phase water-in-oil emulsion (HIPE), the structure comprising a pigment. The method comprising the steps of: i) suspending dry pigment particles in a suspending medium to create a first suspension of the pigment particles; ii) providing an oil phase component blend, the blend comprising at least one monomer; iii) providing an aqueous phase component solution; iv) providing an initiator solution; v) blending the first suspension with the oil phase component blend to create a second suspension of the pigment particles in the oil phase component blend; vi) blending the second suspension with the aqueous phase component solution and the initiator solution in a water:oil ratio of at least 2.85:1, by volume, and sufficient mechanical energy and shear to create a water-in-oil HIPE having a continuous phase comprising the second suspension; and vii) polymerizing monomer components of the oil phase component blend to create an open cell foam structure.

### BRIEF DESCRIPTION OF THE DRAWINGS

FIG. 1 is a plan view of an example of an absorbent article in the form of a feminine hygiene pad, topsheet side facing the viewer.

FIG. 2A is a schematic lateral cross section of the absorbent article of FIG. 1.

FIG. 2B is an enlarged portion 2B of the drawing of FIG. 2A, enlarged to depict sublayers of an absorbent layer, in one example.

FIG. 2C is an enlarged portion 2C of the drawing of FIG. 2A, enlarged to depict sublayers of an absorbent layer, in one example.

FIGS. 3A-3C are plan views of several examples of adhesive deposit patterns within a bonding region, in which a topsheet may be bonded to an absorbent layer.

FIG. 4A is an SEM photograph of a portion of a foam created from a first emulsion as described herein.

FIG. 4B is an SEM photograph of a portion of a foam created from a second emulsion as described herein.

FIG. 4C is an SEM photograph of a portion of a foam created from a third emulsion as described herein.

### DETAILED DESCRIPTION

### Definitions

For purposes herein, the following terms have the following definitions:

“Pigment” means a chemical compound that exists as a solid at 15° C. and 1 atm pressure, has been divided/particularized into powdered form, does not dissolve in or chemically react with any of the liquid components of emulsions identified herein under the conditions described herein, and is recognized in the art as a coloring agent. For purposes herein, “pigment” also includes any of various forms of elemental carbon in powdered form recognized in the art as useful as coloring agents, for example, carbon black.

“Dye” refers to liquid solution containing one or more dissolved compounds (in contrast to undissolved compounds in solid particle form, in suspension) that may be used to impart coloration via exposure/contact, solidification onto and/or reaction with another material.

An “aperture” is an opening of any x-y planar shape profile, which is deliberately formed, punched or cut along a z-direction entirely through a layer, so as to provide a z-direction passageway through the layer.

“Lateral”—with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface. With respect to an absorbent article, the “x-direction” is also the lateral direction.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

“Longitudinal”—with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above. With respect to an absorbent article, the “y-direction” is also the longitudinal direction.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

“x-y plane,” with reference to an absorbent article such as a feminine hygiene pad, or component thereof, when laid out flat on a horizontal surface, means any horizontal plane occupied by the horizontal surface or any layer of the article or component.

“z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms characterizing relative vertical positioning, when used herein to refer to layers, components or other features of an absorbent article such as a feminine hygiene pad, are relative the z-direction and are to be interpreted with respect to the orientation of the article as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in ordinary use that lies closer to the wearer's skin than another feature of the component or structure. For example, a topsheet has a wearer-facing surface that lies closer to the wearer's skin than the opposite, outward-facing surface of the topsheet, when the article is in ordinary use.

With respect to an absorbent article such as a feminine hygiene pad, or a component or structure thereof, “outward-facing” is a relative locational term referring to a feature of the component or structure that when in ordinary use that lies farther from the wearer's skin than another feature of the component or structure. For example, a topsheet has an outward-facing surface that lies farther from the wearer's skin than the opposite, wearer-facing surface of the topsheet, when the article is in ordinary use.

The terms “outboard” and “inboard” are relative terms that characterize the position of a feature of an absorbent article along an x-y plane, relative the longitudinal and lateral axes of the article or relative another feature. The inboardmost point on the article is at the intersection of the longitudinal and lateral axes. The outboardmost points on the article lie along its x-y plane perimeter.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

With respect to a feminine hygiene pad, the terms “front,” “rear,” “forward” and “rearward” relate to features or regions of the pad corresponding to the position it would occupy as ordinarily worn by a user, and the front and rear of the user's body when standing.

### DESCRIPTION

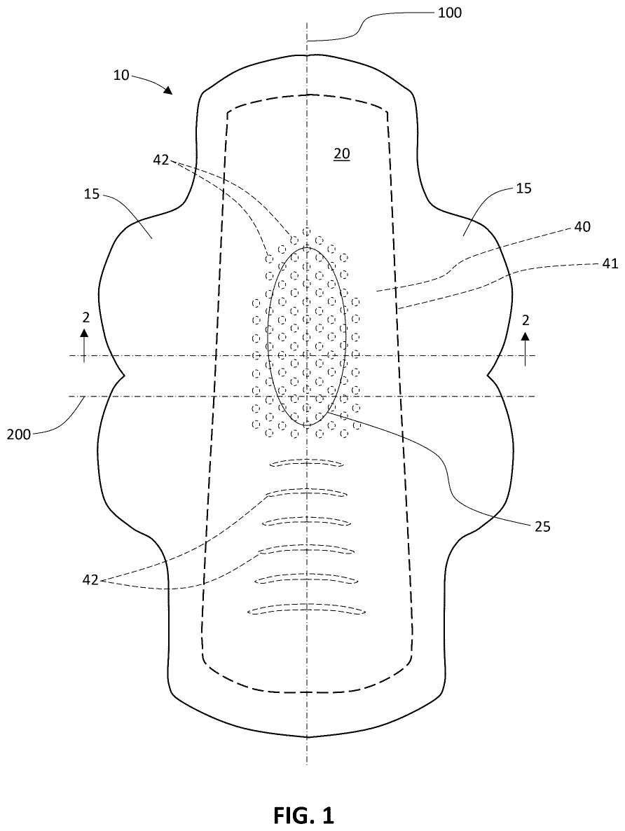

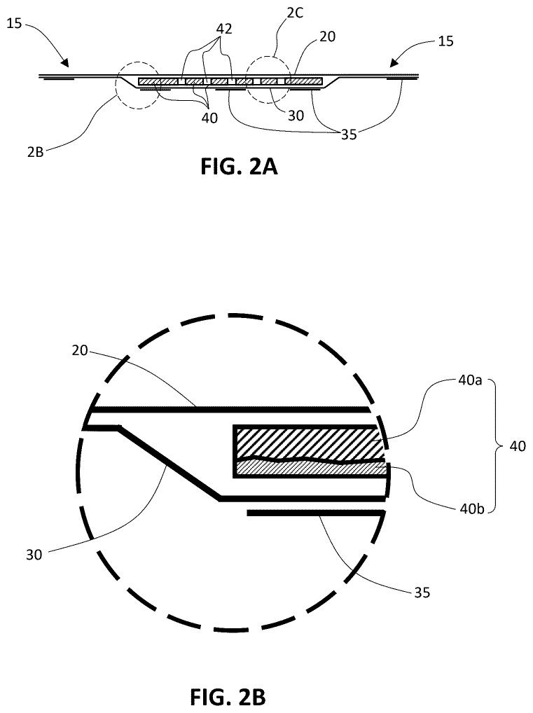

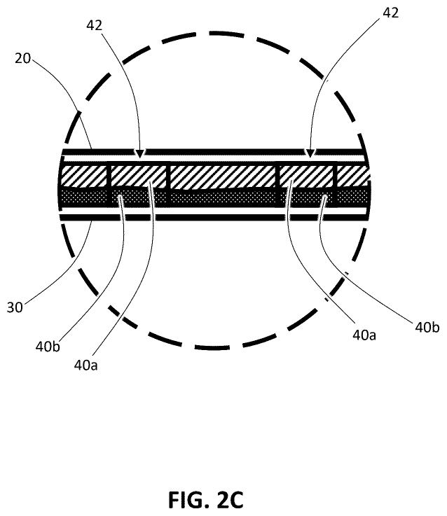

Referring to FIGS. 1 and 2A, an absorbent article such as a feminine hygiene pad 10 may include a liquid permeable topsheet 20, a liquid impermeable backsheet 30 and an absorbent layer 40 disposed between the topsheet and the backsheet. The absorbent layer has an outer perimeter 41. In regions outside the outer perimeter 41, the topsheet and the backsheet may be bonded together in laminated fashion by any suitable mechanism including but not limited to adhesive bonding, thermal bonding, pressure bonding, etc., thereby enveloping and retaining and holding the absorbent layer 40 in place between the topsheet 20 and the backsheet 30. In some configurations, a feminine hygiene pad 10 may include opposing wing portions 15 extending laterally outboard of perimeter 41 by a comparatively greater width dimension than the main portion of the pad. The outer surface of the backsheet 30 forming the undersides of the main portion and the wing portions may have deposits of adhesive 35 thereon. Adhesive deposits 35 may be provided to enable the user to adhere the pad to the inside of her underpants in the crotch region thereof, and wrap the wing portions through and around the inside edges of the leg openings of the underpants and adhere them to the outside/underside of the underpants in the crotch region, providing supplemental holding support and helping guard the leg edges of the underpants against soiling. When feminine hygiene pad 10 is packaged, adhesive deposits 35 may be covered by one or more sheets of release film or paper (not shown) that covers and shields the adhesive deposits 35 from contact with other surfaces until the user is ready to remove the release film or paper and place the pad for use.

### Topsheet

Topsheet 20 may be formed of any suitable liquid permeable nonwoven web material, or alternatively, a suitably liquid permeable (e.g., apertured) film material. Referring back to the figures, the topsheet 20 is positioned adjacent a wearer-facing surface of the absorbent layer 40 and may be joined thereto and to the backsheet 30 by any suitable attachment or bonding method. The topsheet 20 and the backsheet 30 may be joined directly to each other in the peripheral regions outside the perimeter 41 of the absorbent layer 40 and may be indirectly joined by directly joining them respectively to wearer-facing and outward-facing surfaces of the absorbent layer or additional optional layers included with the pad.

The topsheet material may be selected to be or include any material that is comfortably compliant and pliable, soft-feeling, and non-irritating to the wearer's skin. A suitable topsheet material for a feminine hygiene pad will include a liquid permeable material that is comfortable when in contact with the wearer's skin and permits discharged menstrual fluid to rapidly move through it. A suitable topsheet may be made of various materials such as a nonwoven web material, an apertured film, or combination or laminate thereof. Examples of topsheet materials that may be suitable are disclosed in U.S. Pat. Nos. 3,929,135; 4,151,240; 4,319,868; 4,324,426; 4,343,314; 4,591,523; 4,609,518; 4,629,643; 4,695,422; WO 96/00548; U.S. Pat. Nos. 4,950,264; 4,988,344; 4,988,345; 3,978,185; 7,785,690; 7,838,099; 5,792,404; 5,665,452; 8,728,049; 7,553,532; 7,172,801; 8,440,286; 7,648,752; 7,410,683; 7,655,176 or U.S. Pat. Nos. 7,402,723; 8,614,365; 8,704,036; 6,025,535; US 2015/041640; US 2017/0258647; US 2016/0167334; US 2016/0166443; US 2017/0258651; U.S. Pat. Nos. 8,017,534; 4,100,324; US 2003/0200991; U.S. Pat. No. 5,508,102; US 2003/0211802; EP 0 333 228; WO 2009/10938; US 2017/0000695; US 2017/0002486; U.S. Pat. No. 9,944,047; 2017/0022643 US 2018/0002848; U.S. Pat. No. 8,501,646; EP 1 988 793; and US 2007/0275622.

### Absorbent Layer

In some configurations, the absorbent layer 40 may be formed of or include a layer of absorbent open-celled foam material. In some examples, the foam material may include at least first and second sublayers 40a, 40b (FIG. 2B) of absorbent open-celled foam material, the sublayers being in direct face-to-face contact with each other. In such examples, the wearer-facing sublayer may be a relatively larger-celled foam material, and the outward-facing sublayer may be a relatively smaller-celled foam material, for purposes explained in more detail below.

The open-celled foam material may be a foam material that is manufactured via polymerization of the continuous oil phase of a water-in-oil high internal phase emulsion. Exemplary water-in-oil high internal phase emulsions may include those described in US2024/0115436 and US2024/0156647A1, both of which are incorporated by reference herein.

A water-in-oil HIPE has two phases. One phase is a continuous oil phase comprising monomers to be polymerized, and an emulsifier to help stabilize the HIPE. The monomer component may be included in an amount of from about 80% to about 99% by weight of the oil phase. The emulsifier component, which is soluble in the oil phase and suitable for forming a stable water-in-oil emulsion, may be included in the oil phase in an amount of from about 1% to about 20% by weight of the oil phase.

In general, the monomers may include at least one substantially water-insoluble monofunctional alkyl acrylate or alkyl methacrylate. For example, monomers of this type may include C₃-C₁₈ alkyl acrylates and C₄-C₁₈ methacrylates, such as ethylhexyl acrylate, butyl acrylate, hexyl acrylate, octyl acrylate, nonyl acrylate, decyl acrylate, isodecyl acrylate, tetradecyl acrylate, benzyl acrylate, nonyl phenyl acrylate, hexyl methacrylate, 2-ethylhexyl methacrylate, octyl methacrylate, nonyl methacrylate, decyl methacrylate, isodecyl methacrylate, dodecyl methacrylate, tetradecyl methacrylate, and octadecyl methacrylate.

The oil phase may also include from about 2% to about 40%, by weight of the oil phase, a substantially water-insoluble, polyfunctional crosslinking comonomer. Examples of crosslinking monomers of this type can comprise monomers containing two or more activated acrylate, methacrylate groups, or combinations thereof. Examples of this group can include 1,6-hexanedioldiacrylate, 1,4-butanedioldimethacrylate, trimethylolpropane triacrylate, trimethylolpropane trimethacrylate, 1,1 2-dodecyldimethacrylate, 1,14-tetradecanedioldimethacrylate, ethylene glycol dimethacrylate, neopentyl glycol diacrylate (2,2-dimethylpropanediol diacrylate), hexanediol acrylate methacrylate, glucose pentaacrylate, sorbitan pentaacrylate, and the like. Other examples of crosslinkers contain a mixture of acrylate and methacrylate moieties, such as ethylene glycol acrylate-methacrylate and neopentyl glycol acrylate-methacrylate.

Any third substantially water-insoluble comonomer may be added to the oil phase to modify properties of the HIPE foams. In certain cases, “toughening” monomers may be desired to impart toughness to the resulting HIPE foam. These include monomers such as styrene, vinyl chloride, vinylidene chloride, isoprene, and chloroprene. Monomers may also be added to confer flame retardancy, as disclosed, for example, in U.S. Pat. No. 6,160,028. Monomers may be added to impart fluorescent properties; to impart radiation resistance; to impart opacity to radiation (for example lead tetraacrylate); to disperse charge; to reflect incident infrared light; to absorb radio waves; to make surfaces of the HIPE foam struts or cell walls wettable; or for any other desired property in a HIPE foam. In some cases, these additional monomers may slow the overall process of conversion of HIPE to HIPE foam, the tradeoff being necessary if the desired property is to be conferred.

The oil phase may further include an emulsifier to stabilize the HIPE. Emulsifiers used in a HIPE can include: (a) sorbitan monoesters of branched C16-C24 fatty acids; linear unsaturated C16-C22 fatty acids; and linear saturated C12-C14 fatty acids, such as sorbitan monooleate, sorbitan monomyristate, and sorbitan monoesters, sorbitan monolaurate diglycerol monooleate, polyglycerol monoisostearate, and polyglycerol monomyristate; (b) polyglycerol monoesters of -branched C16-C24 fatty acids, linear unsaturated C16-C22 fatty acids, or linear saturated C12-C14 fatty acids, such as diglycerol monooleate (for example diglycerol monoesters of C18:1 fatty acids), diglycerol monomyristate, diglycerol monoisostearate, and diglycerol monoesters; (c) diglycerol monoaliphatic ethers of -branched C16-C24 alcohols, linear unsaturated C16-C22 alcohols, and linear saturated C12-C14 alcohols, and mixtures of these emulsifiers. See U.S. Pat. Nos. 5,287,207 and 5,500,451. Another emulsifier that may be used is polyglycerol succinate, which is formed from an alkyl succinate, glycerol, and triglycerol.

Such emulsifiers, and combinations thereof, may be added to the oil phase so that they constitute about 1% to about 20% by weight of the oil phase. In certain examples, coemulsifiers may also be used to provide additional control of cell size, cell size distribution, and emulsion stability. Examples of coemulsifiers include phosphatidyl cholines and phosphatidyl choline-containing compositions, aliphatic betaines, long chain C12-C22 dialiphatic quaternary ammonium salts, short chain C1-C4 dialiphatic quaternary ammonium salts, long chain C12-C22 dialkoyl(alkenoyl)-2-hydroxyethyl, short chain C1-C4 dialiphatic quaternary ammonium salts, long chain C12-C22 dialiphatic imidazolinium quaternary ammonium salts, short chain C1-C4 dialiphatic imidazolinium quaternary ammonium salts, long chain C12-C22 monoaliphatic benzyl quaternary ammonium salts, long chain C12-C22 dialkoyl(alkenoyl)-2-aminoethyl, short chain C1-C4 monoaliphatic benzyl quaternary ammonium salts, short chain C1-C4 monohydroxyaliphatic quaternary ammonium salts, ditallow dimethyl ammonium methyl sulfate, and combinations thereof.

In some aspects, a photoinitiator may be included in the oil phase and/or aqueous phase. Suitable photoinitiators for use in forming foams within contemplation of the present disclosure may absorb UV light at wavelengths of about 200 nanometers (nm) to about 800 nm, or about 250 nm to about 450 nm. If the photoinitiator is in the oil phase, suitable types of oil-soluble photoinitiators may include benzyl ketals, α-hydroxyalkyl phenones, α-amino alkyl phenones, acylphospine oxides, and combinations thereof. If a photoinitiator is to be included in the aqueous phase, suitable types of water-soluble photoinitiators may include benzophenones, benzils, thioxanthones, and combinations thereof.

The dispersed aqueous phase of a HIPE comprises water, and may also comprise one or more components, such as initiator or electrolyte, wherein in certain aspects, the one or more components are at least partially water soluble.

One component included in the aqueous phase may be a water-soluble electrolyte. The water phase may contain from about 0.2% to about 40% by weight of the aqueous phase of a water-soluble electrolyte. The electrolyte minimizes the tendency of monomers, comonomers, and crosslinkers that are primarily oil soluble to also dissolve in the aqueous phase. Examples of electrolytes may include chlorides or sulfates of alkaline earth metals such as calcium or magnesium and chlorides or sulfates of alkali earth metals such as sodium. Such electrolyte can include a buffering agent for the control of pH during the polymerization, including such inorganic counterions as phosphate, borate, and carbonate, and mixtures thereof.

Another component that may be included in the aqueous phase is a water-soluble free-radical initiator. The initiator can be present at up to about 20 mole percent based on the total moles of polymerizable monomers present in the oil phase. Suitable initiators may include ammonium persulfate, sodium persulfate, potassium persulfate, 2,2′-azobis(N,N′-dimethyleneisobutyramidine)dihydrochloride, azo initiators, redox couples like persulfate-bisulfate, persulfate-ascorbic acid, and other suitable redox initiators.

HIPE foam may be produced from the polymerization of the monomers comprising the continuous oil phase of a HIPE. In come configurations, a HIPE foam layer may have one or more sublayers, and may be either homogeneous or heterogeneous polymeric open-celled foams. Homogeneity and heterogeneity relate to distinct layers within the same HIPE foam, which are similar in the case of homogeneous HIPE foams and differ in the case of heterogeneous HIPE foams. A heterogeneous HIPE foam may contain at least two distinct sublayers that differ with regard to their chemical composition, physical properties, or both; for example, sublayers may differ with regard to one or more of foam density, polymer composition, specific surface area, or pore size (also referred to as cell size). In some configurations, for a HIPE foam if the difference relates to pore size, the average pore size in the respective sublayers may differ by at least about 20%, or by at least about 35%, or by at least about 50%. In other configurations, if the differences in the sublayers of a HIPE foam layer relate to density, the densities of the layers may differ by at least about 20 or by at least about 35%, or by at least about 50%. For instance, if one layer of a HIPE foam has a density of 0.020 g/cc, another layer may have a density of at least about 0.024 g/cc or less than about 0.016 g/cc, or at least about 0.027 g/cc or less than about 0.013 g/cc, or at least about 0.030 g/cc, or less than about 0.010 g/cc. If the differences between the layers are related to the chemical composition of the HIPE or HIPE foam, the differences may reflect a relative amount difference in at least one monomer component, for example by at least about 20%, or by at least about 35%, or by at least about 50%. For instance, if one sublayer of a HIPE or HIPE foam is composed of about 10% styrene in its formulation, another sublayer of the HIPE or HIPE foam may be composed of at least about 12%, or of at least about 15%.

A HIPE foam layer structured to have distinct sublayers formed from differing HIPEs may provide a HIPE foam layer with a range of desired performance characteristics. For example, a HIPE foam layer comprising first and second foam sublayers, wherein the first foam sublayer has a relatively larger pore or cell size, than the second sublayer, when used in an absorbent article may more quickly absorb incoming fluids than the second sublayer. For example, when the HIPE foam layer is used to form an absorbent layer of a feminine hygiene pad, the first foam sublayer may be layered over the second foam sublayer having relatively smaller pore sizes, as compared to the first foam sublayer, which exert more capillary pressure and draw the acquired fluid from the first foam sublayer, restoring the first foam sublayer's ability to acquire more fluid from above. HIPE foam pore sizes may range from 1 to 200 μm, or in some configurations may be less than 100 μm. HIPE foam layers of the present disclosure having two major parallel surfaces may be from about 0.5 to about 10 mm thick, or in some configurations from about 2 to about 10 mm. The desired thickness of a HIPE foam layer will depend on the materials used to form the HIPE foam layer, the speed at which a HIPE is deposited on a belt, and the intended use of the resulting HIPE foam layer.

The HIPE foam layers of the present disclosure are relatively open-celled. This refers to the individual cells or pores of the HIPE foam layer being in substantially unobstructed communication with adjoining cells. The cells in such substantially open-celled HIPE foam structures have intercellular openings or windows that are large enough to permit ready fluid transfer from one cell to another within the HIPE foam structure. For purpose of the present disclosure, a HIPE foam is considered “open-celled” if at least about 80% of the cells in the HIPE foam that are at least 1 μm in size are in fluid communication with at least one adjoining cell.

In some configurations, for example when it is used to form an absorbent layer of a feminine hygiene pad, a HIPE foam layer may be flexible and exhibit an appropriate glass transition temperature (Tg). The Tg represents the midpoint of the transition between the glassy and rubbery states of the polymer. In general, HIPE foams that have a Tg that is higher than the temperature of use can be strong but will also be relatively rigid and potentially prone to fracture (brittle). In some configurations, regions of the HIPE foams of the current disclosure which exhibit either a relatively high Tg or excessive brittleness will be discontinuous. Since these discontinuous regions will also generally exhibit high strength, they can be prepared at lower densities without compromising the overall strength of the HIPE foam.

HIPE foams intended for applications requiring flexibility should contain at least one continuous region having a Tg as low as possible, so long as the overall HIPE foam has acceptable strength at in-use temperatures. In certain examples, the Tg of this region will be less than about 40° C. for foams used at about ambient temperature conditions; in certain other examples Tg will be less than about 30° C. For HIPE foams used in applications wherein the use temperature is higher or lower than ambient temperature, the Tg of the continuous region may be no more than 10° C. greater than the use temperature, or the same as use temperature, or about 10° C. less than use temperature wherein flexibility is desired. Accordingly, monomers are selected as much as possible that provide corresponding polymers having lower Tg's.

HIPE foams useful for forming absorbent layers and/or sublayers within contemplation of the present disclosure, and methods for their manufacture, also include but are not necessarily limited to those foams and methods described in U.S. Pat. Nos. 10,045,890; 9,056,412; 8,629,192; 8,257,787; 7,393,878; 6,551,295; 6,525,106; 6,550,960; 6,406,648; 6,376,565; 6,372,953; 6,369,121; 6,365,642; 6,207,724; 6,204,298; 6,158,144; 6,107,538; 6,107,356; 6,083,211; 6,013,589; 5,899,893; 5,873,869; 5,863,958; 5,849,805; 5,827,909; 5,827,253; 5,817,704; 5,817,081; 5,795,921; 5,741,581; 5,652,194; 5,650,222; 5,632,737; 5,563,179; 5,550,167; 5,500,451; 5,387,207; 5,352,711; U.S. Pat. Nos. 5,397,316; 5,331,015; 5,292,777; 5,268,224; 5,260,345; 5,250,576; 5,149,720; 5,147,345; US 2005/0197414; US 2005/0197415; US 2011/0160326; US 2011/0159135; US 2011/0159206; US 2011/0160321; US 2011/0160689; US 20230075291; WO2022/145237; WO2022/145241; JP2022104703; JP2022104710; JP2022104695; JP2022104696; JP2022104709; CN113307906; CN113289049; CN113429622; CN113603822; CN113980344; CN114230713; CN114395165; CN114835843; CN114410045; CN114957539; and CN114891149, which are incorporated herein by reference to the extent not inconsistent herewith.

### Impartation of Coloration to HIPE Foam

It has been learned that imparting components of an absorbent article, such as a feminine hygiene pad, with elements of coloration, may have signaling effects for the consumer/user/wearer, whereby the consumer associates elements of coloration with issues such as appropriate pad location/placement for use within underwear; efficacy or absorbency of regions or portions of the pad; efficacy or absorbency of particular components of the pad, etc. Such signaling effects may be competitively advantageous to the manufacturer.

Absorbent, open-celled foam manufactured as described above is typically substantially white in color under ordinary lighting conditions, as a result of relative colorlessness of the polymeric material forming the strut structures constituting the foam, combined with reflection and/or refraction of incident light striking the foam in many varying directions.

It is believed, however, that techniques for successfully imparting coloration to absorbent components formed of HIPE have not been developed. Herein, “coloration” refers to coloration that has been imparted during manufacture of the foam by steps occurring prior to its final formation, resulting in coloration that is present within the interior physical structure of the foam—in contrast to surface or superficial coloration imparted by printing or other surface application of, e.g., an ink, after the foam has been manufactured. It is further believed that imparting coloration to absorbent components formed of HIPE foam as described herein has not been specifically contemplated. Herein, success is defined as impartation of coloration via addition of a coloring agent such that the coloring agent (a) does not prevent formation of the HIPE or render it unworkably unstable prior to polymerization; (b) following manufacture of the foam, does not dissolve into aqueous fluid, and thereby bleed or wash out of the foam to any substantial extent, upon contact with the foam by the fluid; and (c) will not be dislodged from the foam structure in solid/particulate form to any substantial and/or unacceptable extent, upon mechanical manipulation of the foam, following manufacture and finish processing of the foam.

It has been learned, however, that an absorbent layer formed of HIPE foam, or in examples in which the HIPE foam has two or more sublayers, that a single sublayer thereof, may be successfully imparted with coloration. In other examples in which the HIPE foam has two or more sublayers, respective sublayers may be imparted with differing intensities of the same color, or even differing colors.

Experimental attempts to impart coloration to HIPE foam using dyes (as defined herein), have proven unsuccessful. It has been found that the dye materials with which experiments have been conducted have been undesirably interactive or reactive with components of the HIPE as identified above, frustrating attempts to create a suitable emulsion, or even changing from or losing their intended visible colors.

On the other hand, experimentation with pigment materials (as defined herein) has proven successful under certain circumstances and conditions. It has been learned that, by suspending solid pigment particles, of an appropriately small average size, in an organic liquid medium that is miscible with the oil components (particularly the monomers) of the HIPE, followed by emulsification to form the HIPE and polymerization of the oil/monomer/continuous phase, causes pigment particles to be at least partially embedded in the resulting solid polymer material that forms the strut structures of the foam. In some examples, the organic liquid used as a medium in which pigment is suspended may be reactive, but it will not react prior to or during emulsification. It may polymerize in the polymerization step following the emulsification step. The pigment particles are typically not soluble or reactive in aqueous fluids such as those contemplated herein (aqueous phase component of the HIPE, and bodily exudates such as menstrual fluid or urine). It is believed that, when sufficiently embedded within the strut structures, the pigment particles are generally not susceptible to being dislodged by mechanical manipulation of the foam structure, or being washed out upon wetting of the structure.

### Example 1

HIPE foams were made with and without blue 15 pigment at room temperature (21° C.), in order to demonstrate the effects of pigment source on the resultant foam structure.

A base oil phase blend containing monomers and emulsifiers as described in US 2024/0115436 was prepared. For the first batch, 149.50 g of the base oil phase blend was added to a 400 ml beaker with 0.38 g dry phthalocyanine blue (Pigment Blue 15) powder, product no. 252980, from MilliporeSigma/Sigma-Aldrich, Inc., St. Louis, Missouri.

The combination was mixed in the beaker for 30 minutes using an IKA Ultra Turrax T18 homogenizer and S 18N-10G dispersing tool (IKA Works, Inc., Wilmington, NC), to create a metastable pigment-in-monomer suspension. The homogenizer was set to 10,000 rpm and the bottom of the tool was positioned 2 mm from the bottom of the beaker.

Forty (40) mL of the resulting first pigment-in-monomer suspension batch was placed into a first 40 mL glass vial.

Thirty minutes later, 40 mL of the base oil phase blend was placed into a second 40 mL glass vial.

Thirty minutes later, a second pigment-in-monomer suspension batch was made. 149.68 g of the base oil blend was added to a 400 mL beaker with 1.48 g of Sun Chemical CROFS5711536 (blue 15 pigment suspension) (Sun Chemical Corporation, Northlake, Illinois). Sun Chemical CROFS5711536 is a suspension of approximately 24 weight percent blue 15 pigment (phthalocyanine blue) compounded with 1-dodecamine, in a blend of organic liquids including ethanol, propyl acetate and ethyl acetate. The combination was mixed for 5 minutes with the S 18N-10G dispersing tool for an IKA Ultra Turrax T18 homogenizer, to create a metastable pigment suspension. The homogenizer was set to 10,000 rpm and the bottom of the tool was positioned 2 mm from the bottom of the beaker. 40 mL of this second pigment-in-monomer suspension batch was placed into a third 40 mL glass vial.

The respective vials containing the first and second pigment-in-monomer suspensions and the base oil blend without pigment were kept at room temperature (21° C.) and visually evaluated for pigment particle sedimentation (settling), one day following their preparation. The vials were labeled as the following: 1=solid pigment particles; 2=base oil without pigment; 3=liquid dispersion of pigments.

After passage of a day it was observed via unaided visual inspection that the first pigment-in-monomer suspension (vial 1) exhibited apparent stratification of blue color intensity from top to bottom of the vial, with the most intense blue coloration appearing at the bottom of the vial, while the liquid above was substantially less intensely colored/substantially more pale in hue. This was believed to evidence substantial sedimentation of relatively larger/heavier pigment particles. Without intending to be bound by theory, it is believed that the pale blue color remaining above the bottom of the vial was imparted by a reduced concentration of smaller/lighter pigment particles remaining in suspension.

The liquid within the vial containing the base oil phase blend without pigment (vial 2) appeared to have a slightly/light yellowish color, both shortly after preparation and transfer to the vial, and after passage of a day.

After passage of a day it was observed via unaided visual inspection that the second pigment-in-monomer suspension (vial 3) exhibited apparent substantial consistency of blue color intensity from top to bottom of the vial. This is believed to evidence substantially less sedimentation, and substantially greater maintenance of the suspension, than occurred in the first pigment-in-monomer suspension. Without intending to be bound by theory, it is believed that the greater maintenance of the pigment suspension evidenced in the second pigment-in-monomer suspension is attributable to the selections of organic suspending liquids, and possibly, other suspension enhancers (such as one or more surface modifiers) present in the commercially available Sun Chemical product identified above. It may also be possible that the Sun Chemical product contained pigment particles having a smaller average size and weight, than the Sigma-Aldrich product, which is believed would make them more easily maintained in suspension. Further, without intending to be bound by theory, it is believed that a relatively small particle size of the suspended pigment, specifically the Sun Chemical CROFS5711536 PB15 suspension (equal to or less than 1 μm), may facilitate effective embedding and/or encapsulation of pigment particles within the struts/strut structures of the open-cell foam, as compared with pigments having particle sizes greater than 1 μm.

### Example 2

Three samples of HIPE foam were polymerized from high internal phase emulsions, produced as follows. One was produced from the base oil phase blend alone, and two were produced from the pigment-in-monomer suspensions described in Example 1 (the oil phase compositions).

An aqueous phase composition and a water-soluble free-radical initiator solution was prepared according to US 2024/0115436.

The oil phase composition was added to a 350 mL polypropylene bottle with an inner diameter of 6 cm, which was placed into a heated (65° C.), double-jacketed beaker. The aqueous phase composition was pumped (at a rate of 59 mL/min) via a heating bath (70° C.), into the bottle. The initiator solution was added dropwise to the bottle following the addition of the aqueous phase composition, over a period of 15 to 20 seconds. An overhead stirrer operating at 200 rpm was used to agitate the bottle contents for 2 minutes following addition of the aqueous phase composition, to emulsify the contents of the bottle. After the stirring tool was removed the bottle containing the emulsion was loosely covered and placed into an oven set to 70° C., and left overnight, which resulted in curing (polymerization of the oil phase). On the following day, the resulting cured, polymeric wet foam was gently removed from the bottle and cut along a horizontal direction relative the upright bottle, into 3 mm thick slices. These slices were flushed/rinsed with deionized water, and then dried.

The first sample emulsion was made using the first pigment-in-monomer suspension (Sigma-Aldrich) from Example 1. After creation of the first pigment-in-monomer suspension, 8 grams of it were added to the polypropylene bottle. Then the stirring tool was lowered into the bottle and the stirrer was activated. As the stirrer was operating, 208 g of the aqueous phase composition and 8 g of initiator were added. After the final 2 minutes of mixing, the stirrer was removed and the bottle of emulsion was placed in the oven for overnight curing, followed by slicing, rinsing/flushing, and drying, as described above.

The second sample emulsion was made using the (unpigmented) base oil blend from Example 1. 8 grams of the base oil blend were added to the polypropylene bottle. Then the stirring tool was lowered into the bottle and the stirrer was activated. As the stirrer was operating, 208 g of the aqueous phase composition and 8 g of initiator were added. After the final 2 minutes of mixing, the stirrer was removed and the bottle of emulsion was placed in the oven for overnight curing, followed by slicing, rinsing/flushing, and drying, as described above.

The third sample emulsion was made using the second pigment-in-monomer suspension (Sun Chemical) from Example 1. After creation of the second pigment-in-monomer suspension, 8 grams of it were added to the polypropylene bottle. Then the stirrer tool was lowered into the bottle and the stirrer was activated. As the stirrer was operating, 208 g of the aqueous phase composition and 8 g of initiator were added. After the final 2 minutes of mixing, the stirrer was removed and the bottle of emulsion was placed in the oven for overnight curing, followed by slicing, rinsing/flushing, and drying, as described above.

The resultant foam slices from the first and third sample emulsions were all medium blue in color to the naked eye. The foam slices from the second sample emulsion were all substantially white in color.

The foam from the first sample emulsion exhibited a lack of colorfastness, consistently shedding blue-colored material when rubbed onto white paper. The foam from the third emulsion did not exhibit such shedding/rub off tendency, and exhibited substantially better colorfastness.

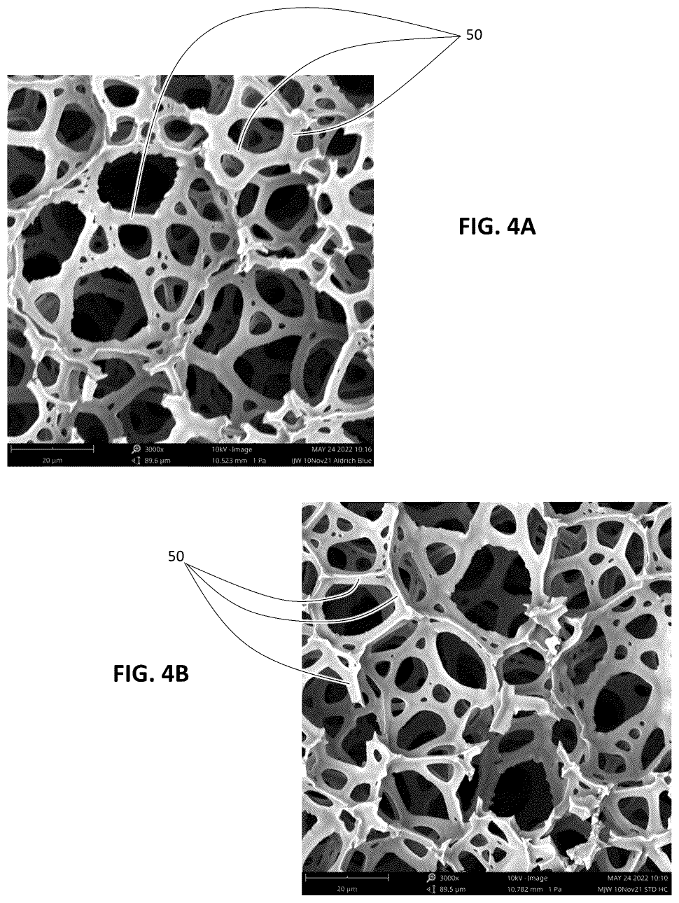

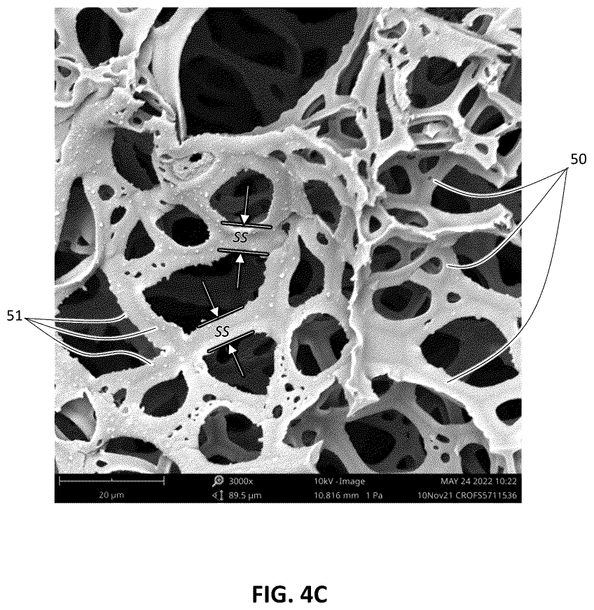

FIG. 4A is an SEM photograph of a portion of the foam created from the first sample emulsion (Sigma-Aldrich pigment). FIG. 4B is an SEM photograph of the foam created from the second (unpigmented) sample emulsion. FIG. 4C is an SEM photograph of the foam created from the third sample emulsion (Sun Chemical pigment). In each of the photos in FIGS. 4A-4C, a network of struts 50, framing windows in adjacent cells, and forming the open-celled foam structure, can be seen.

The SEM micrographs illustrate that the foam generated from the third sample emulsion (FIG. 4C) exhibited a number of small bumps 51 on the surfaces of the struts 50. Without intending to be bound by theory, it is believed that these bumps 51 are pigment particles that are at least partially embedded in the polymerized material forming the struts. In contrast, such bumps are not visible on the foam struts 50 created from the first (FIG. 4A) or second (FIG. 4B) sample emulsions.

Without intending to be bound by theory, it is believed that organic liquids in which the pigment particles were dispersed and suspended in the Sun Chemical product used to make the second pigment-in-monomer suspension, were miscible with the components of the base oil blend, and effectively carried the pigment particles into and helped suspend them in the oil phase, where they remained during and after polymerization, whereby they became embedded and thereby affixed within or to the polymer strut structure of the resulting foam. In contrast, it is believed that the dry pigment particles (Sigma-Aldrich) blended into the first pigment-in-monomer suspension did not effectively remain uniformly dispersed and/or suspended within the oil phase in sufficient proportion through emulsification. Following emulsification and drying, many of these particles were merely deposited within the open cells of the foam structure without being embedded and affixed within or to the struts. As a result, they were subject to being dislodged from the foam structure upon mechanical stress thereto.

Without intending to be bound by theory, it is believed that suitable organic liquids that are miscible with acrylate monomers contemplated herein include methanol, ethanol, isopropanol, an acrylate, a methacrylate, an acetate, and combinations thereof.

Suitable pigment dispersants/suspending liquids and surface modifiers include surfactants and polymeric dispersants containing urethanes, polyamides, polyesters, polyethers, styrenes, and combinations thereof. Additional reactive dispersants include materials containing acrylate or methacrylate oligomers and those containing additional co-polymers such as epoxy acrylate and/or methacrylate, polyester acrylate and/or methacrylate, urethane acrylate and/or methacrylate, aliphatic acrylate and/or methacrylate, aromatic acrylate and/or methacrylate, and combinations thereof. The reactive acrylate or methacrylate oligomer may comprise at least 2 functional groups, or 12 or less functional groups, or 6 or less than functional groups, or about 4 or less functional groups, or from about 2 to about 6 functional groups, reciting for said range every 1 increment therein. In some aspects, oligomer(s) may comprise a glass transition temperature of 150° C. or less, or 125° C. or less, or 100° C. or less.

Additionally, it is observed that the organic liquid components of the Sun Chemical pigment material, in addition to being miscible with most of the oil components of the HIPE, if not polymerized with the other oil components, had sufficient volatility to cause them to evaporate from the HIPE and/or polymerized foam, during processing, such that they did not remain in any substantial quantity following manufacture of the foam, and/or, at least in the case of ethanol (which is miscible with water), remaining quantities were susceptible to being washed out in the rinsing step. Further, the identified liquid components of the Sun Chemical pigment material are materials that are deemed safe for use in producing consumer products such as the absorbent products contemplated herein. For purposes herein a “safe” material is one that is recognized by applicable U.S. regulatory bodies (having jurisdiction) as safe for use in manufacturing, or inclusion in, finished products, or are not prohibited by such regulatory bodies for use in manufacturing, or from inclusion in, finished products, that are externally-worn, disposable personal absorbent hygiene products (i.e., disposable diapers, absorbent pants, feminine hygiene pads/sanitary napkins).

From the foregoing, it is deduced that a suspension of pigment in one or more organic liquids that are miscible with the oil phase components of a HIPE may be introduced and blended with the oil phase components prior to emulsification, followed by formation of the HIPE emulsion and then polymerization, to result in a successfully-colored HIPE foam.

Further, from the visible bumps 51 on the struts shown FIG. 4C it can be deduced that it is preferable that the mean size of the pigment particles suspended in the organic liquid(s) be substantially smaller than the mean size of the struts 50. It is preferable that the mean size of the pigment particles be no greater than 50% of the mean strut size, more preferably no greater than 25% of the mean strut size, even more preferably no greater than 10% of the mean strut size, and still more preferably no greater than 6% of the mean strut size. Alternatively, for HIPE foams of the type contemplated herein, it is preferable for the mean pigment particle size to be no greater than 8,000 nm, more preferably no greater than 4,000 nm, even more preferably no greater than 1,600 nm, and still more preferably no greater than 1,000 nm.

For purposes herein, the size of an individual pigment particle is its largest linear dimension and a mean pigment particle size value for a given pigment supply may be obtained by sampling and measuring using any suitable sampling techniques and magnification and measurement aids, or alternatively, may be provided by the supplier of the pigment material, as a specification for the material.

For purposes herein a “strut” is any solid structure or portion thereof that is present between two immediately adjacent windows; and the size SS of an individual strut 50 is its largest linear dimension, measured along a direction orthogonal to that portion of its length between the immediately adjacent windows that it lies between—at the location where the windows are closest together. See, e.g., FIG. 4C (dimension SS). It will be understood that a mean strut size SS may be obtained via taking measurements of randomly selected struts shown in facing position, in scaled SEM photographs of randomly selected foam samples, to obtain a statistically sound mean value.

It has been specifically discovered that a suspension of phthalocyanine blue (or blue 15) pigment may be suitable, although it is believed and contemplated that pigments of other colors may be suitable as well. Without intending to be bound by theory, it is believed that pigments selected from the group consisting of Pigment Blue (PB) 15 (including PB 15:1, PB 15:2, PB 15:3, blue 15:4, and PB 15:6); black 6 (carbon black); black 7; green 7; violet 19; violet 23, and combinations thereof, are suitable. However, it has been learned that the shade or hue imparted by blue 15 may be perceived particularly favorably by consumers/wearers, as connoting cleanliness or sanitariness, signaling absorbency or other efficacy or positive product attributes, and not visually interactive in esthetically unpleasing ways with coloration(s) resulting from absorbance of bodily exudates (e.g., urine, fecal matter and/or menstrual fluid) and staining thereby, for absorbent articles of the types contemplated herein. In other configurations, however, a shade or hue imparted by powdered elemental carbon (e.g., carbon black), i.e., a shade of gray, may also be suitable and advantageous. In still other configurations, a blend of blue 15 and elemental carbon may be desired for a perceived deeper/darker/grayish-blue hue.

Additional pigments that may be suitable include those having the following COLOR INDEX classifications: Pigment Green (PG) 7 and 36; Pigment Orange (PO) 5, PO16, PO34, PO36, PO60, PO62, PO64, PO66, and PO67; Pigment Magenta (PM) and Pigment Red (PR) 101, PR112, PR122, PR146, PR149, PR170, PR176, PR178, PR179, PR181, PR185, PR187, PR188, PR200, PR202, PR207, PR208, PR214, PR220, PR224, PR242, PR251, PR254, PR255, PR260, PR264, PR48:1, PR48:2, PR48:3, PR52, PR52:1, and PR57:1; Pigment Violet (PV) 19, PV23, PV31, and PV37; Pigment Yellow (PY) 12, PY13, PY14, PY17, PY74, PY83, PY120, PY138, PY139, PY155, PY151, PY168, PY175, PY179, PY180, PY181 and PY185; Pigment Blue (PB) 15, PB15:1, PB15:2, PB15:3, PB15:4, PB15:6; Pigment Black (PBl) 2, PBl5 and PBl7; Carbon Black; Titanium Dioxide (including rutile and anatase); Zinc Oxide, Zinc Sulfide, and combinations thereof. COLOR INDEX classifications are published by the Association of Textile Chemists and Colorists and the Society of Dyers and Colourists.

In some aspects, the pigments may be chosen from phthalocyanine pigments, oxazine pigments, quinacridone pigments, diarylide pigments, Bis-Azo and Mono-Azo pigments, naphthol pigments, imidazoline pigments, isoindoline pigments, perylene pigments or combinations thereof.

For ranges of coloration of foam that are believed to be pleasing to consumers of absorbent articles of the types contemplated herein, while not requiring an unnecessarily high pigment load level, or one that cannot be successfully integrated into the foam material, it may be desired to select pigment type(s) and adjust the pigment load so as to achieve a coloration of the foam material having CIELAB color space values in the following ranges:
L*≥50, more preferably ≥75, more preferably ≥85;
128≥a*≥−128, more preferably 75≥a*≥−128; and

$$0 \geq {b}^{*} \geq - 128 .$$

The CIELAB color space values for a sample of foam may be obtained by gently removing the foam layer from the article in which it is a component, cutting a suitable sample therefrom, and determining its CIELAB color space values by applying the method described in U.S. Pat. No. 11,173,070.

### Absorbent Article

As reflected in FIG. 1, an absorbent layer formed of HIPE foam may include one or more patterns of perforations 42, including at least a first pattern disposed within an expected discharge location (coincidental with bonding region 25) overlying the intersection of longitudinal and lateral axes 100, 200 of the pad. Perforations 42 may be punched, cut or otherwise formed through the entire z-direction depth of the HIPE foam absorbent layer, or only through a wearer-facing layer or partially into the wearer-facing portion thereof. When a HIPE foam absorbent layer is disposed in direct contact with a topsheet as described herein, with no intervening acquisition layer formed of another material, perforations 42 may serve as a group of “reservoirs” to receive, temporarily hold, and aid in distributing rapid discharges of relatively small quantities of menstrual fluid, until the HIPE foam has sufficient time to distribute and absorb the fluid via capillary action. Additionally, such perforations help decrease bending stiffness of the absorbent layer, which may help increase comfort of the pad for the wearer. A pattern of perforations having an average radius or other largest dimension of 1.0 mm to 4.0 mm, and more preferably 1.5 mm to 3.5 mm may be included, within, for example, the area occupied by the bonding region 25. The pattern may include perforations at a numerical density of 3.0 to 9.0 perforations per cm², and more preferably 4.0 to 8.0 perforations per cm². In selecting the appropriate average size, numerical density, and surface area occupied by the pattern of perforations, the manufacturer may wish to balance the volume of the “reservoirs” desired with the need to retain absorbent material in locations proximate to and about the expected discharge location. Additional details concerning configurations of such perforations in combination with examples of suitable absorbent layers may be found in U.S. Pat. No. 8,211,078.

The absorbent layer formed of HIPE foam should be imparted with sufficient CWPA (described below) to have capability to effectively draw discharged fluid from a topsheet over a time of use/wear of the pad during menstruation that is normal and expected for feminine hygiene pads, for example, from 4 to 8 hours. As noted below, the CWPA of a material is in part affected by its volume. Thus, it may be desired that an absorbent layer 40 formed of HIPE foam have a caliper (prior to wetting) that provides satisfactory volume to a standard-sized pad. Of course, a relatively thick pad can be manufactured, but that is typically deemed undesirable for daytime use in view of desires for flexibility/pliability and thinness, for comfort and discreetness under clothing. The manufacture must balance these competing objectives. Accordingly a feminine hygiene pad with a HIPE foam absorbent layer as contemplated herein, it may be desired that the layer have a caliper in the majority of its wearer-facing surface area (prior to wetting) of 1 mm to 5 mm, or more preferably 1.5 mm to 3.5 mm, or even more preferably 2.0 mm to 3.0 mm. (The caliper of a HIPE foam layer may be measured visually, with assistance of magnification/microscopy and/or photography or any other facilitating techniques and equipment, to any extent deemed useful.) Where the absorbent layer 40 includes two sublayers 40a, 40b as described herein, it may be desired that the upper sublayer 40a have a caliper (prior to wetting) of 0.64 mm to 3.2 mm, or preferably 0.96 mm to 2.24 mm, or even more preferably 1.28 mm to 1.92 mm; and it may be desired that the lower sublayer 40b have a caliper (prior to wetting) of 0.16 mm to 0.80 mm, or more preferably 0.24 mm to 0.56 mm, or even more preferably 0.32 mm to 0.48 mm.

It is contemplated that a HIPE foam material used to form an absorbent layer 40 of an absorbent article may be imparted with coloration as described herein. It is further contemplated that a HIPE foam absorbent layer 40 having two sublayers (40a, 40b) as described herein may be manufactured, in which only one of the two sublayers is pigmented as described herein, while the other sublayer is left substantially white. In some configurations, it may be desired to pigment a lower, or outward facing, sublayer 40b of a HIPE foam absorbent layer 40 and leave the upper, or wearer-facing, sublayer 40a substantially white. In such an configuration, referring to FIGS. 1, 2A and 2C, through a translucent topsheet 20, the consumer/user can see the coloration of the outward-facing layer 40b along the perimeter 41 edges, and on the interior surfaces of any apertures 42 that may be cut through the foam absorbent layer as described herein. The consumer/user may perceive that the coloration of a lower/outward-facing sublayer 40b signals something different about that layer (which in some examples, would be the case, where the lower/outward facing layer 40b has differing features such as smaller average cell sizes and greater absorbency). This may be particularly beneficial in examples of feminine hygiene pads which have perforations 42 in a longitudinally and/or laterally centralized region such as a central bonding region 25 as described and depicted herein. The centralized region coincides in whole or in part with a location of expected fluid discharge or insult, when the pad is suitably located/placed within the underpants and worn by a user. In such examples, through a translucent topsheet the user can partially view the inside surfaces of the perforations 42 and perceive that a lower/outward-facing sublayer 40b of the absorbent layer 40 is imparted with color, and thereby receive a signal that the sublayer 40b has differing (e.g., greater) absorbency properties than the upper/wearer-facing sublayer 40a. Because the lower/wearer-facing sublayer 40b is disposed deeper within the article away from the user, the user may perceive a signal that fluid is being drawn down, distributed, absorbed and retained relatively deeply within the article, away from the user's skin and upper surfaces of the pad. Such signal may provide comfort to the user and increase confidence that the pad will function as desired.

In other examples and for similar signaling and/or esthetic purposes, pigment loads may be varied between the respective sublayers, to result in differing levels of color intensity/hue between the sublayers. Similarly, in other examples, differing pigment types may be selected for the respective sublayers, such that the respective sublayers will be imparted with differing colors, for example, carbon black to impart a gray hue to one sublayer, and blue 15 to impart a blue hue to another sublayer.

In combination with imparting coloration to a foam layer and/or one or more sublayers as described above, it may be desired that the backsheet selected have sufficient translucency (sufficiently low opacity) to enable a viewer to perceive coloration of the foam layer therethrough. For such purposes, it may be desired that the backsheet have an opacity no greater than 50 percent.

It is further believed that some consumers may be unaware that an absorbent foam layer in an absorbent may have a plurality of sublayers, particularly when the foam layer as a whole is relatively thin. Accordingly, it is believed that such consumers will gain understanding that a product has multiple sublayers when one sublayer has imparted coloration or hue differing from coloration or hue of the other sublayer(s). Alternatively, one sublayer that has a different color pattern than the other sublayer(s) could be distinguished by consumers.

It is further believed for absorbent articles that feel relatively dry on a wearer-facing surface even when loaded with bodily exudate, and also have effective stain masking or concealing capability, that coloration of an absorbent layer or sublayer can facilitate user understanding that there is a multilayer foam present, facilitate user understanding concerning the manner in which the product works/absorbs, thus, facilitate user understanding as to when the multilayer product is fully saturated with exudate and should be replaced.

### Absorbency Properties and Interface Between Topsheet and Absorbent Layer

In configurations in which the topsheet is formed of a hydrophilic and absorbent web material, the topsheet material may tend to retain fluid on its wearer-facing and outward-facing surfaces, and within the interstitial spaces between and along the surfaces of the fibers of the web material, unless the underlying material has absorption capacity and absorption pressure greater than the desorption pressure of the topsheet, and there is sufficient direct contact maintained between the topsheet and the underlying absorbent layer to enable the fluid to move from fiber surfaces within the topsheet structure, directly to surfaces of material within the underlying absorbent layer structure, such that the underlying absorbent layer may draw the fluid from the topsheet. Prior to the time it is fully saturated, an absorbent material will not release absorbed fluid unless an adjacent material with greater affinity for the fluid is in sufficient direct contact. Accordingly, it is important to provide structure sufficient to maintain sufficient contact, without obstructing fluid movement. No intervening layer or structure of material, or at least no intervening layer or structure of material less absorbent that that of the absorbent layer, should be interposed between the material of the topsheet 20 and the material of the absorbent layer 40, at least within the bonding region 25, more preferably over a majority of the wearer-facing surface area of the absorbent layer 40, and even more preferably over the entirety of the wearer-facing surface area of the absorbent layer 40—unlike systems provided in many current feminine hygiene pads, which include a distinct fluid acquisition/distribution material layer between the topsheet and the absorbent materials of the absorbent core.

In some examples, sufficient direct contact between the topsheet 20 and the absorbent layer 40 may be effected by deposit(s) of adhesive between the topsheet and the absorbent layer, adhesively bonding them in close z-direction proximity. The adhesive may be applied in a pattern or arrangement of adhesive deposits interspersed with areas in which no adhesive is present (unbonded areas), such that the adhesive holds the two layers in close z-direction proximity, while areas remain in which no adhesive is present to obstruct z-direction fluid movement between the layers.

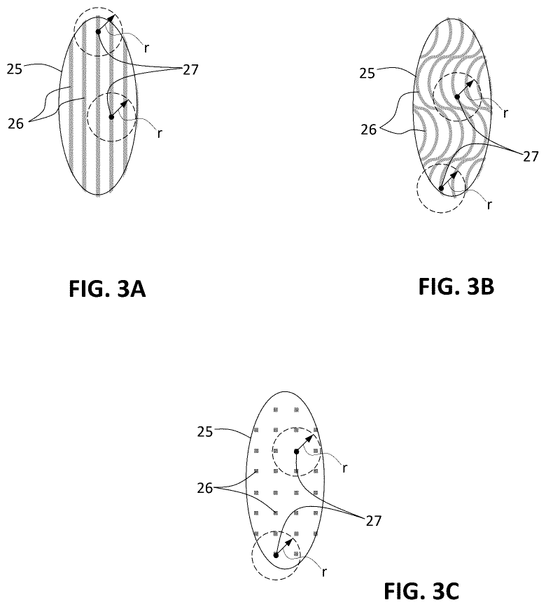

Referring to FIGS. 1 and 3A-3C, to ensure that the topsheet and absorbent layer are held in sufficiently close z-direction proximity at least in the area of the topsheet expected to receive a discharge of fluid, it may be desired to dispose a bonding region 25 on the pad at a location that includes the intersection of the longitudinal and lateral axes 100, 200. The bonding region 25 should be of sufficient size to be reliably present beneath the expected discharge location when the pad is in use, with reasonably minor variations of placement by the wearer within the underpants; accordingly, it may be desired that the bonding region have an area of at least 15 cm², more preferably at least 30 cm². Even more preferably, it may be desired that the bonding region have an area that is at least half of the total wearer-facing surface area of the absorbent layer. (Note: FIGS. 3A-3C are not presented herein as actual size or scale depictions.)

To ensure that the topsheet 20 and the absorbent layer remain in sufficient z-direction proximity during use, it may be desired that, within any identifiable first point location 27 within the bonding region, at which the topsheet is bonded to the absorbent layer, there is a second point location at which the topsheet is bonded to the absorbent layer, within a 10 mm radius, more preferably within a 6 mm radius, 5 mm radius, 4 mm radius, and even more preferably with a 3 mm radius r of the first point location. Referring to FIGS. 3A-3C, depicting three non-limiting examples, it can be seen that a variety of patterns or arrangements of bonds (via adhesive deposits 26 or other bonding mechanisms) may be employed to impart this feature. Within radius r of each point location 27, there are a number of additional point locations where bonding between the topsheet and the absorbent layer is present in the examples depicted.

It will be appreciated that a continuous deposit of adhesive may be applied to bond the topsheet and the absorbent layer within the entirety of bonded region 25, but that such a continuous deposit of adhesive could form a barrier that may obstruct the movement of fluid from the topsheet to the absorbent layer. Accordingly, it is preferable that, in configurations in which the bonding mechanism is deposits of adhesive, the deposits are disposed in a pattern or arrangement that is discontinuous or intermittent such that it creates bonded areas interspersed with unbonded areas between the topsheet and the absorbent layer. Additionally, when the absorbent layer is formed of an open-celled foam (such as a HIPE foam contemplated herein) it may be desired that the adhesive selected not effect adhesion to the absorbent layer via chemical, dispersive or diffusive adhesion with the foam layer at the adhesive deposit locations, but rather, that it effect adhesion to the foam layer mechanically, by flowing to a limited extent into the cells, at least partially assuming the shapes thereof, and solidifying in such position to form mechanical interlocks with the cell structures, which enable the adhesive to hold the topsheet to the absorbent layer. Such an adhesive may be preferred so as not to alter the molecular structure or composition of the foam material, potentially negatively affecting its fluid absorption properties or mechanical strength. In one example, a suitable adhesive for use with a HIPE foam may be H1750 hot melt adhesive from Bostik, Wauwatosa, Wisconsin (currently a subsidiary of Arkema, Columbes, France).

Unapertured topsheets for feminine hygiene pads formed of nonwoven web material and including or consisting predominately of hydrophilic fibers are known and have been included with some feminine hygiene products to date. (Herein, an “unapertured” nonwoven topsheet is one in which a majority of its surface area has not been subjected to any process that creates an arrangement of holes or apertures entirely therethrough, that persist prior to wetting of the topsheet, of an average size (greatest dimension) greater than 0.5 mm along an x-y planar direction.) Although favored by some consumers for their pleasant feel against the skin, topsheets formed of hydrophilic nonwoven web material have been disfavored by other consumers as a result of their substantial absorbency, i.e., capillary absorption and desorption pressures, causing them to resist drainage by conventionally included acquisition/distribution and absorbent layer structures. Following a discharge of menstrual fluid, a pad with such a topsheet overlying a conventional absorbent structure can feel to the user like a wet cloth held against the skin for an extended time period, which many users find objectionable. This dilemma has been present for many years, and to the inventor's knowledge, has not previously been satisfactorily addressed.

It has been discovered, however, that an unapertured hydrophilic fiber topsheet overlaid in direct, sufficient face-to-face proximate relationship with a HIPE foam absorbent layer or other layer adapted/manufactured to have capillary absorption capability sufficient to draw fluid from the topsheet, without any intervening less absorbent layers and in combination with other structural features as described herein, will be substantially drained of fluid by the absorbent layer, and regain a much drier feel against the skin following a discharge. It has been discovered that a suitably composed and manufactured HIPE foam absorbent layer as described herein, for example, has a greater affinity for menstrual fluid than such a topsheet, and thereby, has the capability to draw and retain fluid away from the topsheet when the two are disposed and held in sufficiently effective proximate, contacting relationship with each other. When the absorbent layer has a sufficient volume, it can serve this function over a reasonably suitable time of use of the pad.

An additional purpose, however, is served by maintaining such close spatial proximity between the topsheet and the HIPE foam absorbent layer: The close spatial proximity maximizes the likelihood that a viewer of the article will be able to readily visually perceive coloration imparted to the HIPE foam absorbent layer, through the topsheet.

### Backsheet

The backsheet 30 may be positioned adjacent an outward-facing surface of the absorbent layer 40 and may be joined thereto by any suitable attachment methods. For example, the backsheet 30 may be secured to the absorbent layer 40 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent layer 40 is not joined directly to the backsheet 30.

The backsheet 30 may be impervious, or substantially impervious, to liquids (e.g., urine, menstrual fluid) and may be manufactured from a thin plastic film, although other flexible liquid impervious materials may also be used. As used herein, the term “flexible” refers to materials which are compliant and will readily conform to the general shape and contours of the human body. The backsheet 30 may prevent, or at least substantially inhibit, fluids absorbed and contained within the absorbent layer 40 from escaping and reaching articles of the wearer's clothing which may contact the pad 10 such as underpants and outer clothing. However, in some instances, the backsheet 30 may be made and/or adapted to permit vapor to escape from the absorbent layer 40 (i.e., the backsheet is made to be breathable), while in other instances the backsheet 30 may be made so as not to permit vapors to escape (i.e., it is made to be non-breathable). Thus, the backsheet 30 may comprise a polymeric film such as thermoplastic films of polyethylene or polypropylene. A suitable material for the backsheet 30 is a thermoplastic film having a thickness of from about 0.012 mm (0.5 mil) to about 0.051 mm (2.0 mils), for example. Any suitable backsheet known in the art may be utilized with the present invention.

Some suitable examples of backsheets are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523, 3,989,867, 3,156,242; WO 97/24097; U.S. Pat. Nos. 6,623,464; 6,664,439 and 6,436,508.

The backsheet may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489; 4,341,216; 4,713,068; 4,818,600; EP 203 821; EP 710 471; EP 710 472; and EP 0 793 952.

### Opacity Measurement

The measured opacity of a material is a reflection of the degree to which the material blocks passage of light therethrough. A higher opacity value indicates a higher degree of light blockage by the material. Opacity by contrast ratio is measured using a 0 degree illumination/45 degree detection circumferential optical geometry spectrophotometer with adjustable apertures capable of making CIE color measurements using XYZ coordinates. An example of a suitable spectrophotometer is the Labscan XE with a computer interface running Universal Software (available from Hunter Associates Laboratory Inc., Reston, VA), or equivalent. All testing is performed in a room maintained at a temperature of 23° C.±2° C. and a relative humidity of 50%±2% and samples are conditioned under these same environmental conditions for at least 2 hours prior to testing.

Obtain a sample by removing it from an absorbent article, if necessary. When excising the sample from an absorbent article, use care to not impart any contamination or distortion to the sample layer during the process. The sample is obtained from an area free of folds or wrinkles, and it must be larger than the aperture used on the spectrophotometer. Obtain a sufficient quantity of the sample such that, ideally, five measurements can be made on non-overlapping areas of an individual test material. If the material to be tested has a small area, alternatively, five separate samples may be used. Determine which side of the sample is intended to face the wearer in use.

To measure Opacity, calibrate and standardize the instrument per the vendor instructions using the standard white and black tiles provided by the vendor. Set the spectrophotometer to use the CIE XYZ color space with a D65 standard illumination, a 100 observer, a 1.2 inch aperture with an area view of 1.0, and the UV filter set to nominal. Place the sample centered over the aperture with the wearer-facing side facing the aperture. Place the standard white tile behind the sample, take a reading and record the Y value as Ywhite backing to the nearest 0.01 units. Without moving the sample, remove the standard white tile and replace it with the black standard tile. Take a reading and record the Y value as Yblack backing to the nearest 0.01 units. Calculate Opacity by dividing the Yblack backing value by the Ywhite backing value and then multiply by 100. Record Opacity to the nearest 0.1 percent.

In like fashion, repeat for a total of five measurements on non-overlapping areas of the sample, or alternatively on five separate samples of the same test material. Calculate the arithmetic mean for Opacity obtained from all ten measurements and report to the nearest 0.1 percent.

### Combinations

Paragraph 1. An open-cell foam comprising a structure of interconnected struts formed of polymeric material and defining open cells, resulting from polymerization of a continuous phase of a high internal phase water-in-oil emulsion, the struts having pigment particles at least partially encapsulated or embedded within the polymeric material thereof.
Paragraph 2. The foam of Paragraph 1, wherein the pigment particles are present in combination with a surface modifier.
Paragraph 3. The foam of Paragraph 1 or 2, wherein the pigment particles are chosen from Pigment Blue (PB) 15 (including PB15:1, PB15:2, PB15:3, PB15:4, PB15:6); black 6 (carbon black); black 7; green 7; violet 19; violet 23, or combinations thereof.
Paragraph 4. The foam of any of Paragraphs 1-3, wherein the pigment particles have a mean pigment particle size to be no greater than 8,000 nm, more preferably no greater than 4,000 nm, even more preferably no greater than 1,600 nm, and still more preferably no greater than 1,000 nm.
Paragraph 5. The foam of any of Paragraphs 1-4, wherein pigment particles together with any surface modifier are present in an amount from 0.01 weight percent to 2 weight percent of the structure, more preferably from 0.05 weight percent to 1.5 weight percent of the structure, and even more preferably from 0.10 weight percent to 1.0 weight percent of the structure.
Paragraph 6. The foam of any of Paragraphs 1-5, wherein the polymerized continuous phase comprises polymerized acrylate and/or methacrylate.
Paragraph 7. The foam of any of Paragraphs 1-6, wherein the foam has a Sauter mean diameter cell size between 1 micron and 300 micrometers.
Paragraph 8. The foam of any of Paragraphs 1-7 having the form of a layer with two generally oppositely-disposed planar surfaces and at least first and second sublayers, the first and second sublayers differing from each other in physical characteristics.
Paragraph 9. The foam of Paragraph 8, wherein where first and second sublayers have differing average cell sizes.
Paragraph 10. The foam of Paragraph 8 or 9, wherein only one of the first and second sublayers comprises a predominant weight proportion of the at least partially encapsulated or embedded pigment particles, and the other of the first and second sublayers comprises a minor weight proportion of the at least partially encapsulated or embedded pigment particles, or substantially lacks partially encapsulated or embedded pigment particles.
Paragraph 11. The foam of Paragraph 8 or 9, wherein the first and second sublayers each comprise the at least partially encapsulated or embedded pigment particles, with differing weight proportions of pigment particles between the first and second sublayers.
Paragraph 12. The foam of Paragraph 8 or 9, wherein the first and second sublayers each comprise the at least partially encapsulated or embedded pigment particles, with differing types of pigment particles between the first and second sublayers.
Paragraph 13. An absorbent article (10) comprising a liquid permeable topsheet (20), liquid impermeable backsheet (30), and an absorbent layer (40) disposed between the topsheet and the backsheet, wherein the absorbent layer comprises the foam of any of Paragraphs 1-8.
Paragraph 14. The absorbent article of Paragraph 13, wherein the absorbent article is a feminine hygiene pad.
Paragraph 15. The absorbent article of Paragraph 13 or 14, wherein the absorbent layer (40) comprises a plurality of sublayers (40a, 40b), each sublayer comprising an open-cell foam comprising a structure of interconnected struts formed of polymeric material and defining open cells, resulting from polymerization of a continuous phase of a high internal phase water-in-oil emulsion, wherein fewer than all sublayers of the plurality of sublayers, comprises the foam of any of Paragraphs 1-8.
Paragraph 16. The absorbent article of Paragraph 15, wherein the open-cell foam comprised by a first of the plurality of sublayers has an average cell size that is relatively larger than that of the open-cell foam comprised by a second of the plurality of sublayers; and the open-cell foam comprised by the second of the plurality of sublayers has an average cell size that is relatively smaller than that of the open-cell foam comprised by the first of the plurality of sublayers.
Paragraph 17. The absorbent article of Paragraph 16, wherein the first of the plurality of sublayers comprises the foam of any of Paragraphs 1-12.
Paragraph 18. The absorbent article of Paragraph 17, wherein the second of the plurality of sublayers comprises the foam of any of Paragraphs 1-12.
Paragraph 19. The absorbent article of any of Paragraphs 15-18, wherein the absorbent layer comprises only two of said sublayers, the respective open-cell foams of said two sublayers being disposed respectively superadjacent and subjacent to, and in direct contact with, each other.
Paragraph 20. A pigment-in-monomer suspension comprising pigment particles and a surface modifier disposed on surfaces of the pigment particles, the surface modifier comprising a cation terminated acrylate oligomer and/or a cation terminated methacrylate oligomer, wherein the pigment particles are suspended in an organic liquid.
Paragraph 21. A pigment-in-monomer suspension comprising pigment particles and comprising one or more monomers selected from the group consisting of C₃-C₁₈ alkyl acrylates and C₄-C₁₈ methacrylates, urethane oligomers, polyamide oligomers and combinations thereof; and one or more organic liquids that are miscible with the one or more monomers.
Paragraph 22. A pigment-in-monomer suspension comprising pigment particles and a surface modifier disposed on surfaces of the pigment particles, wherein the surface modifier comprises polymerizable endgroups that enable it to be bound to a polymer formed via polymerization of the one or more monomers.
Paragraph 23. The pigment-in-monomer suspension of any of Paragraphs 20-21, wherein the organic liquid is selected from the group consisting of methanol, ethanol, isopropanol, 2-Butoxyethanol acetate, any organic polymerizable suspending liquid, such as an acrylate, a methacrylate, and combinations thereof, and preferably, an acrylate or methacrylate.
Paragraph 24. The pigment-in-monomer suspension of Paragraph 21 comprising a surface modifier disposed on surfaces of the pigment particles.
Paragraph 25. The pigment-in-monomer suspension of Paragraph 24, wherein the surface modifier comprises a cation terminated acrylate oligomer and/or a cation terminated methacrylate oligomer.
Paragraph 26. The pigment-in-monomer suspension of any of Paragraphs 20-25, wherein the organic liquid is selected from the group consisting of methanol, ethanol, isopropanol, an acrylate, a methacrylate, propyl acetate, ethyl acetate, and combinations thereof.
Paragraph 27. The pigment-in-monomer suspension of any of Paragraphs 20-26, wherein the pigment particles are chosen from Pigment Blue (PB) 15 (including PB15:1, PB15:2, PB15:3, PB15:4, PB15:6); black 6 (carbon black); black 7; green 7; violet 19; violet 23, and combinations thereof.
Paragraph 28. The pigment-in-monomer suspension of any of Paragraphs 20-27 wherein the pigment particles have a mean pigment particle size no greater than 8,000 nm, more preferably no greater than 4,000 nm, even more preferably no greater than 1,600 nm, and still more preferably no greater than 1,000 nm.
Paragraph 29. A method for manufacturing an open cell foam structure comprising a polymerized continuous phase of a high internal phase water-in-oil emulsion (HIPE), the structure comprising pigment, the method comprising the steps of:
   i) suspending dry pigment particles in a suspending medium to create a first suspension of the pigment particles;
   ii) providing an oil phase component blend, the blend comprising at least one monomer;
   iii) providing an aqueous phase component solution;
   iv) providing an initiator solution;
   v) blending the first suspension with the oil phase component blend to create a second suspension of the pigment particles in the oil phase component blend;
   vi) blending the second suspension with the aqueous phase component solution and the initiator solution in water:oil ratio of at least 2.85:1, more preferably at least 25:1 by volume, and sufficient mechanical energy and shear to create a water-in-oil HIPE having a continuous phase comprising the second suspension; and
   vii) polymerizing monomer components of the oil phase component blend to create an open cell foam structure.
Paragraph 30. The method of Paragraph 29, wherein the suspending medium consists of one or more organic liquids that are miscible with the at least one monomer.
Paragraph 31. The method of Paragraphs 29 or 30, wherein the monomer is selected from the group consisting of C₃-C₁₈ alkyl acrylates and C₄-C₁₈ methacrylates, and combinations thereof.
Paragraph 32. The method of Paragraphs 29 or 30, wherein the suspending medium is selected from the group consisting of methanol, ethanol, isopropanol, an acrylate, a methacrylate, propyl acetate, ethyl acetate, and combinations thereof.
Paragraph 33. The method of any of Paragraphs 29-32, wherein the pigment particles are chosen from Pigment Blue (PB) 15 (including PB15:1, PB15:2, PB15:3, PB15:4, PB15:6); black 6 (carbon black); black 7; green 7; violet 19; violet 23, and combinations thereof.
Paragraph 34. The method of any of Paragraphs 29-33, wherein the pigment particles have a mean pigment particle size no greater than 8,000 nm, more preferably no greater than 4,000 nm, even more preferably no greater than 1,600 nm, and still more preferably no greater than 1,000 nm.
Paragraph 35. The method of any of Paragraphs 29-34, wherein the monomer comprises an acrylate or methacrylate.
Paragraph 36. The method of any of Paragraphs 29-35, wherein the oil phase component blend comprises a cross-linker.
Paragraph 37. The method of any of Paragraphs 29-36, wherein the oil phase component blend comprises an emulsifier.
Paragraph 38. An absorbent article comprising a liquid permeable topsheet, liquid impermeable backsheet, and an absorbent layer disposed between the topsheet and the backsheet, wherein the absorbent layer comprises an open cell foam structure manufactured according to the method of any of examples 29-37.
Paragraph 39. The absorbent article of example 38, wherein the absorbent article is a feminine hygiene pad.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety to the extent not inconsistent herewith and unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An open-cell foam comprising a structure of interconnected struts formed of polymeric material and defining open cells, resulting from polymerization of a continuous phase of a high internal phase water-in-oil emulsion, the struts having pigment particles at least partially encapsulated or embedded within the polymeric material thereof.

2. The foam of claim 1, wherein the pigment particles comprise a surface modifier.

3. The foam of claim 2, wherein the surface modifier comprises a cation terminated acrylate oligomer and/or a cation terminated methacrylate oligomer.

4. The foam of claim 1, wherein the pigment particles are chosen from phthalocyanine pigments, oxazine pigments, quinacridone pigments, diarylide pigments, bis-azo pigments, mono-azo pigments, naphthol pigments, imidazoline pigments, isoindoline pigments, perylene pigments, or combinations thereof.

5. The foam of claim 1, wherein the pigment particles have a mean pigment particle size of no greater than about 8,000 nm.

6. The foam of claim 2, wherein pigment particles together with the surface modifier are present in an amount of from about 0.01 weight percent to about 2 weight percent of the structure.

7. The foam of claim 1, wherein the polymerized continuous phase comprises polymerized acrylate and/or methacrylate.

8. The foam of claim 1, where the foam has a Sauter mean diameter cell size of between about 1 micron and about 300 micrometers.

9. The foam of claim 1 having the form of a layer with two generally oppositely-disposed planar surfaces and at least a first sublayer and a second sublayer, the first and second sublayers differing from each other in physical characteristics.

10. The foam of claim 9, wherein the first and second sublayers have differing average cell sizes.

11. The foam of claim 9, wherein only one of the first and second sublayers comprises a predominant weight proportion of the at least partially encapsulated or embedded pigment particles, and the other of the first and second sublayers comprises a minor weight proportion of the at least partially encapsulated or embedded pigment particles, or substantially lacks partially encapsulated or embedded pigment particles.

12. The foam of claim 9, wherein the first and second sublayers each comprise the at least partially encapsulated or embedded pigment particles, with differing weight proportions of pigment particles between the first and second sublayers.

13. The foam of claim 9, wherein the first and second sublayers each comprise the at least partially encapsulated or embedded pigment particles, with differing types of pigment particles between the first and second sublayers.

14. An absorbent article comprising a liquid permeable topsheet, a liquid impermeable backsheet, and an absorbent layer disposed between the topsheet and the backsheet, wherein the absorbent layer comprises the foam of claim 1.

15. The absorbent article of claim 14, wherein the absorbent article is a feminine hygiene pad.

16. A method for manufacturing an open cell foam structure comprising a polymerized continuous phase of a high internal phase water-in-oil emulsion (HIPE), the structure comprising a pigment, the method comprising the steps of:
i) suspending dry pigment particles in a suspending medium to create a first suspension of the pigment particles;
ii) providing an oil phase component blend, the blend comprising at least one monomer;
iii) providing an aqueous phase component solution;
iv) providing an initiator solution;
v) blending the first suspension with the oil phase component blend to create a second suspension of the pigment particles in the oil phase component blend;
vi) blending the second suspension with the aqueous phase component solution and the initiator solution in a water:oil ratio of at least 2.85:1, by volume, and sufficient mechanical energy and shear to create a water-in-oil HIPE having a continuous phase comprising the second suspension; and
vii) polymerizing monomer components of the oil phase component blend to create an open cell foam structure.

17. The method of claim 16, wherein the suspending medium consists of one or more organic liquids that are miscible with the at least one monomer.

18. The method of claim 17, wherein the one or more organic liquids is selected from the group consisting of methanol, ethanol, isopropanol, an acrylate, a methacrylate, propyl acetate, ethyl acetate, and combinations thereof.

19. The method of claim 16, wherein the pigment particles have a mean pigment particle size of no greater than about 8,000 nm.

20. The method of claim 16, wherein the monomer comprises an acrylate or methacrylate.
